(19) United States
(12) Patent Application Publication
(10) Pub. No.: US 2008/0099007 A1
(43) Pub. Date: May 1, 2008

(54) FOOTWARMER

(76) Inventor: Sheng-sun Shih, Taipei (TW)

Correspondence Address: ROBERT W. BECKER & ASSOCIATES, 707 HIGHWAY 333, SUITE B, TIJERAS, NM, 87059-7507, US

(21) Appl. No.: 11/924,120

(22) Filed: Oct. 25, 2007

(30) Foreign Application Priority Data
Oct. 25, 2006 (DE) 20 2006 016 270.4

(51) Int. Cl. A61F 7/08

(52) U.S. Cl. 126/204

(57) ABSTRACT

A footwarmer having at least one interspace formed by an inner material and an outer material and filled with a granular filling embodied as a cushion-like insert that can be inserted into or removed from the at least one interspace through an opening.

The instant application should be granted the priority date of 25 Oct. 2006, the filing date of the corresponding German patent application DE 20 2006 016270.4.

### BACKGROUND OF THE INVENTION

The present invention relates to a footwarmer.

A shoe-like footwarmer is described in DE 20 2006 007 638 U1, and contains a granular filling comprised of a linseed/lavender mixture.

Such footwarmers are used by first heating them in an oven or microwave. The footwarmers are then placed on the feet so as to warm them.

The drawback of such known designs is that the granular filling cannot be removed. Thus, the known footwarmers are not suitable for washing.

It is therefore an object of the present application to provide a washable footwarmer.

### BRIEF DESCRIPTION OF THE DRAWINGS

This object, and other objects and advantages of the present invention, will appear more clearly from the following specification in conjunction with the accompanying schematic drawings, in which:

FIG. 1 is a plan view of one exemplary embodiment of a footwarmer;

FIG. 2 is a side view of the footwarmer of FIG. 1; and

FIG. 3 shows the footwarmer of FIG. I with the zippers opened and the inserts removed.

### SUMMARY OF THE INVENTION

The footwarmer of the present application comprises at least one interspace that is formed by an inner material and an outer material and is filled with a granular filling embodied as a cushion-like insert, which can be inserted into or removed from the at least one interspace through an appropriate opening.

A basic concept of the present application is the design of the granular filling as cushion-like or small sack-like inserts. These inserts can be removed and introduced through an opening of the interspace. Pursuant to a preferred embodiment, the material of the footwarmer is a washable textile material, so that the footwarmer can be washed without the inserts, thus improving hygiene. The casing of the insert can be made of a thin textile material.

Pursuant to an advantageous embodiment, the opening of the interspace can be closed off by a closure device, in particular a zipper, Velcro-type closure or button closure.

The footwarmer can be provided with a plurality of unconnected interspaces, whereby for each interspace, a shape-adapted insert, which is intended to largely fill the interspace, can be provided.

Pursuant to a preferred embodiment, the footwarmer has an interspace in the region of the sole. The opening for this interspace can be disposed in a side portion of the footwarmer. The footwarmer can additionally be provided with an interspace in the region of the instep, whereby the appropriate opening can also be disposed in this region.

Further specific features of the present invention will be described in detail subsequently.

### Description of Specific Embodiments

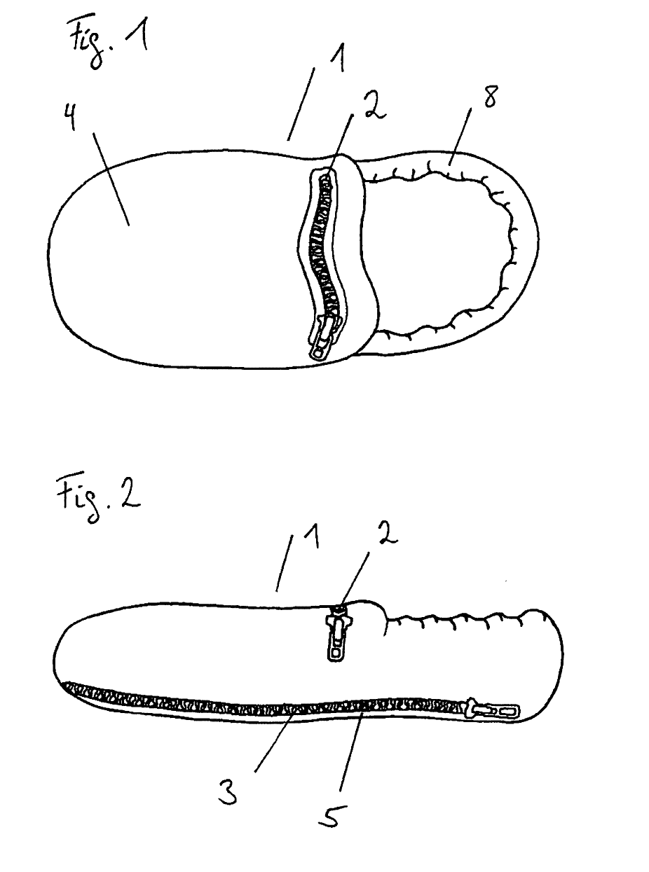

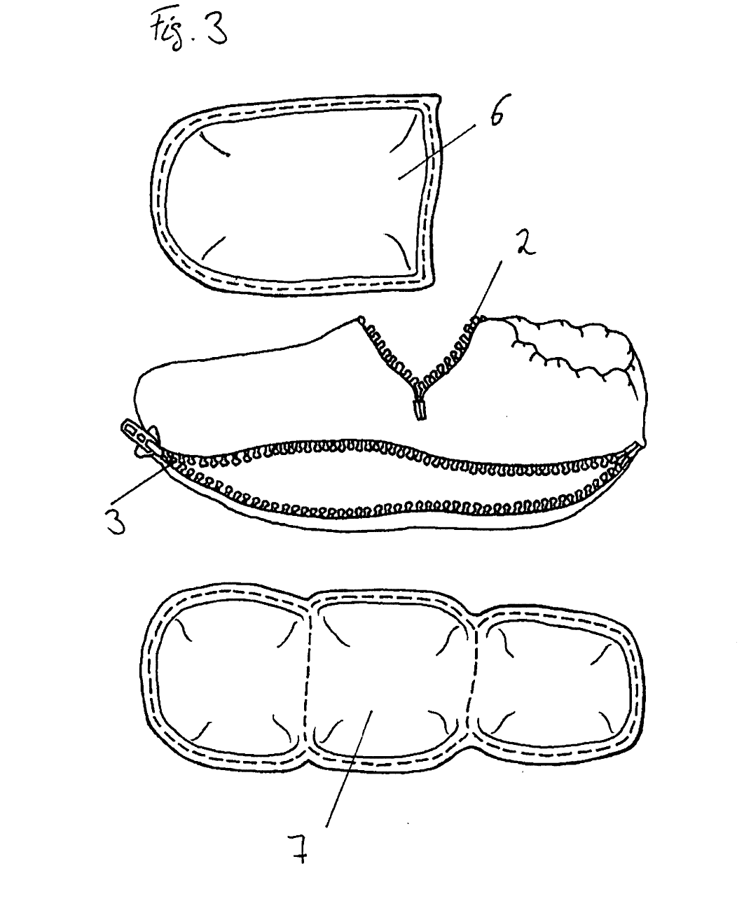

Referring now to the drawings in detail, FIGS. 1 and 2 show a shoe-like footwarmer 1 that is made of a washable fleece or cotton material, and that has a closed heel. The fit on a person's foot is ensured by a sewn-in rubber or elastic cord 8. In the region of the instep 4 and of the sole 5, the footwarmer 1 has interspaces or interstitial spaces that are formed by an inner material and an outer material, and in which are disposed a granular filling. The filling of granular material can be comprised of linseed (flaxseed), although the present invention is not limited to this material. With other shoe shapes, the interspaces can also be disposed at other locations. The inner material and the outer material are made of varied materials.

FIG. 3 shows that the granular filling is embodied as cushion-like inserts 6 and 7 having sewn edges. The casing in which the granular filling is disposed is comprised of a thin textile material, for example cotton.

The insert 6 can be introduced into one interspace through an opening that can be closed off, for example by means of the zipper 2. The insert 7, which can be introduced into a further interspace through an opening that can closed off, for example by means of the zipper 3, is divided into three individual chambers so that when a person walks in the footwarmers, the granular filling cannot shift toward one end or side.

The specification incorporates by reference the disclosure of German priority document DE 20 2006 016270.4 filed 25 Oct. 2006.

The present invention is, of course, in no way restricted to the specific disclosure of the specification and drawings, but also encompasses any modifications within the scope of the appended claims.

What I claim is:

1. A footwarmer comprising at least one interspace, wherein said at least one interspace is formed by an inner material and an outer material and is filled with a granular filling, further wherein said granular filling is embodied as a cushion-like insert, and wherein said insert is adapted to be inserted into or removed from said at least one interspace through an opening.

2. A footwarmer according to claim 1, wherein said inner material and said outer material are comprised of washable textile material.

3. A footwarmer according to claim 1, wherein said opening is adapted to be closed by a closure device.

4. A footwarmer according to claim 3, wherein said closure device is a zipper, a Velcro-type closure, or a button closure.

5. A footwarmer according to claim 1, and comprising a plurality of interspaces, wherein a respective insert is disposed in each of said interspaces.

6. A footwarmer according to claim 1, wherein an insert is disposed in the region of a sole of said footwarmer.

7. A footwarmer according to claim 6, wherein said opening for said insert is disposed in a side portion of said footwarmer.

8. A footwarmer according to claim 1, wherein an insert is disposed in the region of an instep of said footwarmer.
